(19) United States
(12) Patent Application Publication
(10) Pub. No.: US 2005/0004639 A1
(43) Pub. Date: Jan. 6, 2005

(54) Medical lead with resorbable material

(75) Inventor: John H. Erickson, Plano, TX (US)

Correspondence Address: DOCKET CLERK, DM/ANSI, P.O. BOX 802432, DALLAS, TX, 75380, US

(73) Assignee: Advanced Neuromodulation Systems, Inc., Plano, TX (US)

(21) Appl. No.: 10/613,675

(22) Filed: Jul. 3, 2003

(51) Int. Cl. A61N 1/05

(52) U.S. Cl. 607/122

(57) ABSTRACT

An implantable lead, and method of inserting same in a body, and system for stimulation. The lead includes a first lead and a second lead having a connecting member coupled therebetween. The connecting member maintains the first lead and second lead at a first position relative to each other during insertion of the lead and maintains the first lead and second lead at a second position relative to each other after insertion. In one embodiment, the connecting member is constructed of resorbable material to allow non-surgical removal of the lead(s) when desired.

### TECHNICAL FIELD OF THE INVENTION

The present invention relates to electrical leads, and in particular, an electrical lead for use in the medical field.

### BACKGROUND

Implantable leads having electrodes are used in a variety of applications, including the delivery of electrical stimulation to surrounding tissue, neural or otherwise, as well as measuring electrical energy produce by such tissue. Some leads include lumens for the delivery of other elements, including chemicals and drugs. Whether in a stimulation, sensing or element delivery capacity, such leads are commonly implanted along peripheral nerves, within the epidural or intrathecal space of the spinal column, and around the heart, brain, or other organs or tissue of a patient.

Generally, several elements (conductors, electrodes and insulation) are combined to produce a lead body. A lead typically includes one or more conductors extending the length of the lead body from a distal end to a proximal end of the lead. The conductors electrically connect one or more electrodes at the distal end to one or more connectors at the proximal end of the lead. The electrodes are designed to form an electrical connection or stimulus point with tissue or organs. Lead connectors (sometimes referred to as contacts, or contact electrodes) are adapted to electrically and mechanically connect leads to implantable pulse generators or RF receivers (stimulation sources), or other medical devices. An insulating material typically forms the lead body and surrounds the conductors for electrical isolation between the conductors and for protection from the external contact and compatibility with a body.

Such leads are typically implanted into a body at an insertion site and extend from the implant site to the stimulation site (area of placement of the electrodes). The implant site is typically a subcutaneous pocket that receives and houses the pulse generator or receiver (providing a stimulation source). The implant site is usually positioned a distance away from the stimulation site, such as near the buttocks or other place in the torso area. In most cases, the implant site (and insertion site) is located in the lower back area, and the leads may extend through the epidural space (or other space) in the spine to the stimulation site (middle or upper back, or neck or brain areas). Usually, two or more leads are implanted and one or more electrical paths exist between electrodes of two separately located lead ends.

Current lead designs have different shapes, such as those commonly known as percutaneous and paddle-shaped leads. Paddle leads, which are typically larger than percutaneous leads, are directional and often utilized due to the varying epidural spaces and desired stimulus effect on the tissues or areas. However, current paddle-shaped leads require insertion using surgical means, and hence, removal through surgical means.

Percutaneous leads are typically smaller. When inserted into an epidural space, a percutaneous lead fails to take into account the epidural space during insertion. That is, when multiple percutaneous leads are inserted, the lead ends must be adjusted precisely within the epidural space at the desired location to maintain both orientation and spacing from each other. Since it is generally desirable to insert the leads using as small an insertion tube/site as possible, inserting leads having a fixed structure that maintains a fixed spacing between the leads is undesirable, and most likely not feasible, unless the leads are surgically implanted. Therefore, insertion of such percutaneous leads may be required using surgical means, and hence, removal, through surgical means.

Other prior art percutaneous leads and methods of insertion include different structures or mechanisms that deploy from the leads (or deploy the leads) after the leads are positioned at the stimulation site (e.g., deployed out of the insertion needle/tube). These deployable leads provide a relatively fixed spacing between the leads and orientation as desired. However, these structures or mechanisms that deploy the leads are permanent, and significantly increase the effective size of the leads at the stimulation site. This provides a problem when it is desirable to remove the leads. In such cases, a surgical procedure, such as lamenectomy, must be performed to remove the leads. This limits the removal procedure to surgery.

Accordingly, there exists a need for small leads that account for the space during insertion, are deployable at the stimulation site, and are able to be removed through non-surgical means.

### SUMMARY

According to the present invention, there is provided a lead with a first lead body having at least one electrode and with a second lead body having at least one electrode. A connection member coupled to the first lead body and the second lead body is operable when the connecting member is in a first state to maintain at least a portion of the first lead body in a first position relative to at least a portion of the second lead body.

In another embodiment of the present invention, there is provided a lead system having a first lead, a second lead and means coupled to the first lead and the second lead for maintaining at least a portion of the first lead in a first position relative to at least a portion of the second lead.

In yet another embodiment of the present invention, there is provided a lead system having a first lead, a second lead and a connection member. The connection member includes a first portion attached to the first lead, and a second portion attached to the second lead and coupled to the first portion. At least one of the first portion and the second portion comprises resorbable material.

In another embodiment as described generally in the foregoing paragraph, the connection member includes a third portion coupled to the first portion and the second portion, and at least one of the first portion, the second portion and the third portion comprises resorbable material.

In another embodiment of the present invention, there is provided a method of inserting and positioning a medical lead within a body. The method includes inserting a distal end of a lead into a human body, wherein the lead includes a first lead body, a second lead body, and a connection member. After inserting the lead into the human body, the connection member is disengaged to allow the connection member to maintain the first lead body and the second lead body in a first position with respect to each other.

In still another embodiment of the present invention, there is provided a method of manufacturing a lead. The method includes providing a first lead body having a distal end and a second lead body having a distal end. The distal end of the first lead body is coupled to the distal end of the second lead body with a connection member having a portion thereof comprising resorbable material.

In one embodiment of the present invention, there is provided a system for stimulating a portion of a body. The system includes a source for generating a stimulus and an implantable lead having a first lead, a second lead and a connection member. The connection member includes a first portion attached to the first lead, a second portion attached to the second lead, and a third portion coupled to the first portion and the second portion. At least one of the first portion, the second portion and the third portion comprises resorbable material.

### BRIEF DESCRIPTION OF THE DRAWINGS

For a more complete understanding of the present invention, and the advantages thereof, reference is now made to the following descriptions taken in conjunction with the accompanying drawings, wherein like numbers designate like objects, and in which:

FIG. 1 is a perspective view of a prior art percutaneous lead;

FIG. 2A is a perspective view of one embodiment of a lead in accordance with the present invention;

FIG. 2B is a perspective view of another embodiment of a lead in accordance with the present invention;

FIG. 3A illustrates a portion of the lead of the present invention prior to insertion of the lead into a body;

FIG. 3B illustrates a portion of the lead of the present invention after insertion and properly positioned or deployed within the body;

FIG. 3C illustrates another embodiment of a portion of the lead of the present invention prior to insertion of the lead into a body;

FIG. 4 illustrates one embodiment of a system for stimulation in accordance with the present invention; and

FIG. 5 illustrates another embodiment of a system for stimulation in accordance with the present invention.

### DETAILED DESCRIPTION OF THE INVENTION

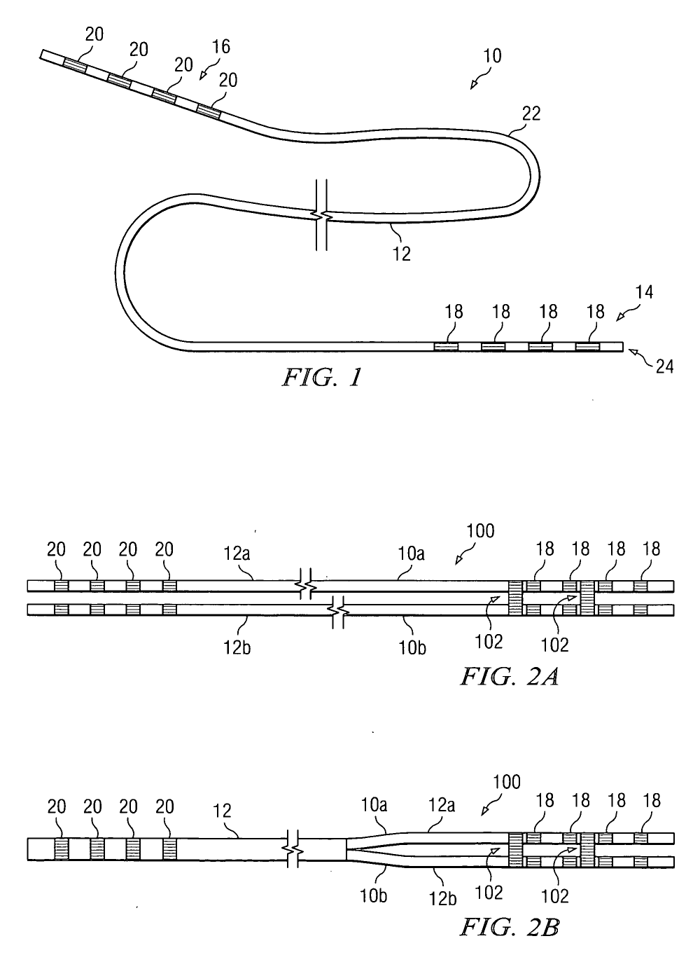

With reference to FIG. 1, there is illustrated an embodiment of a prior art lead 10. The lead 10 includes a distal end 14 and a proximal end 16. The lead 10 includes a lead body 12 that extends from the distal end 14 to the proximal end 16. The distal end 14 of the lead 10 is shown including four band electrodes 18. The proximal end 16 of the lead 10 is shown including four contact electrodes (or ring electrodes) 20 that form a lead connector. The lead 10 generally includes one or more conductors (not shown) extending a substantial portion of the lead 10 to electrically connect the contact electrodes 20 to respective band electrodes 18. An optional lumen 24 is shown that extends through the lead 10 and may be used for different purposes, including the delivery of chemicals or drugs, or for guidance.

As will be appreciated, any number of conductors (not shown), electrodes 18 and contact electrodes 20 may be utilized, as desired. For purposes of illustration only, the lead 10 is shown with four contact electrodes 20 and four electrodes 18. It will be further understood that the distal end 14 of the lead 10 is shown with band electrodes 18. Other types, configurations and shapes of electrodes may be utilized as known to those skilled in the art. Likewise, other types, configurations and shapes of contact electrodes (and lead connectors) may be used, as desired.

Typically, the lead body 12 is a flexible structure (although,not flexible in the longitudinal direction) having a round cross-section. Alternatively, the cross-section of the lead body 12 may be configured in any number of cross-sectional shapes appropriate for the specific application. The figures and following description generally refer to a round cross-sectional shape for the lead body 12 for illustrative purposes only. The lead body generally includes a lead body insulator 22 configured to insulate the conductors and present a biocompatible external surface to the body tissue. In one embodiment, the lead body insulator 22 is coextensive with the conductors.

The lead body insulator 22 is formed of insulating material typically selected based upon biocompatibility, biostability and durability for the particular application. The insulator material may be silicone, polyurethane, polyethylene, polyamide, polyvinylchloride, PTFT, EFTE, or other suitable materials known to those skilled in the art. Alloys or blends of these materials may also be formulated to control the relative flexibility, torqueability, and pushability of the lead 10. Depending on the particular application, the diameter of the lead body 12 may be any size, though a smaller size is more desirable for neurological and myocardial mapping/ablation leads and neuromodulation and stimulation leads.

The conductors may take the form of solid wires, drawn-filled-tube (DFT), drawn-brazed-strand (DBS), stranded wires or cables, ribbons conductors, or other forms known or recognized to those skilled in the art. The composition of the conductors may include aluminum, stainless steel, MP35N, platinum, gold, silver, copper, vanadium, alloys, or other conductive materials or metals known to those of ordinary skill in the art. The number, size, and composition of the conductors will depend on the particular application for the lead 10, as well as the number of electrodes.

The conductors may be configured along the lead body 12 in a straight orientation or spirally or helically wound about the lumen 24 or center of the lead body 12. The conductors are typically insulated from the lumen 24, from each other, and from the external surface of the lead 10 by the insulative material 22. The insulative material 22 may be of single composition, or multiple layers of the same or different materials.

At least one electrode 18 is positioned at the distal end 14 of the lead body 12 for electrically engaging a target tissue or organ. In addition, at least one contact electrode 20 is positioned at the proximal end 16 of the lead body 12 for electrically connecting the conductor(s) to a stimulating or receiving source. In one embodiment, the lead 10 is generally configured to transmit an electric signal from an electrical source (see FIGS. 4 and 5) for application at, or proximate, a spinal nerve or peripheral nerve.

The electrodes 18 and contact electrodes 20 are typically made of a conductive material such as platinum, gold, silver, platinum-iridium, stainless steel, MP35N, or other conductive materials, metals or alloys known to those skilled in the art. The size of the electrodes 18 are generally chosen based upon the desired application. The contact electrodes 20 generally have a size and configuration appropriate to connect the lead 10 to a desired electrical source or receiver.

With reference to FIG. 2A, there is illustrated one embodiment of a lead system 100 in accordance with the present invention. The lead system 100 includes a first lead 10a (or lead body) and a second lead 10b (or lead body). The leads 10a, 10b are the same or similar to the prior art lead 10 illustrated in FIG. 1. The lead system 100 includes a connection (or connecting) member 102 that couples the first lead 10a to the second lead 10b. The connection member 102 functions to bind or attach the first lead 10a to the second lead 10b for insertion through a needle at the insertion site. The connection member 102 also functions to maintain the orientation of the leads 10a, 10b with respect to each other and/or the distance between the leads 10a, 10b. In one embodiment, the connection member 102 is positioned proximate the distal ends—the ends of where the electrodes 18 are positioned.

As will be appreciated, one or more connection member 102 (FIG. 2A illustrates two connection members) may be placed along the lead body 12a, 12b of the lead 100 to couple the leads 10a, 10b and function as described above.

With reference to FIG. 2B, there is illustrated another embodiment of the lead 100 in accordance with the present invention. The lead 100 illustrated in FIG. 2B includes unitary proximal end. The lead body 12 diverges, or splits, into the two leads 10a, 10b, with lead bodies 12a, 12b. The lead 100 includes the same or similar connection (or connecting) member 102 as shown in FIG. 2A. One or more connection member 102 (FIG. 2B illustrates two connection members) may be placed along the lead body 12a, 12b of the lead 100 to couple the leads 10a, 10b and function as described above.

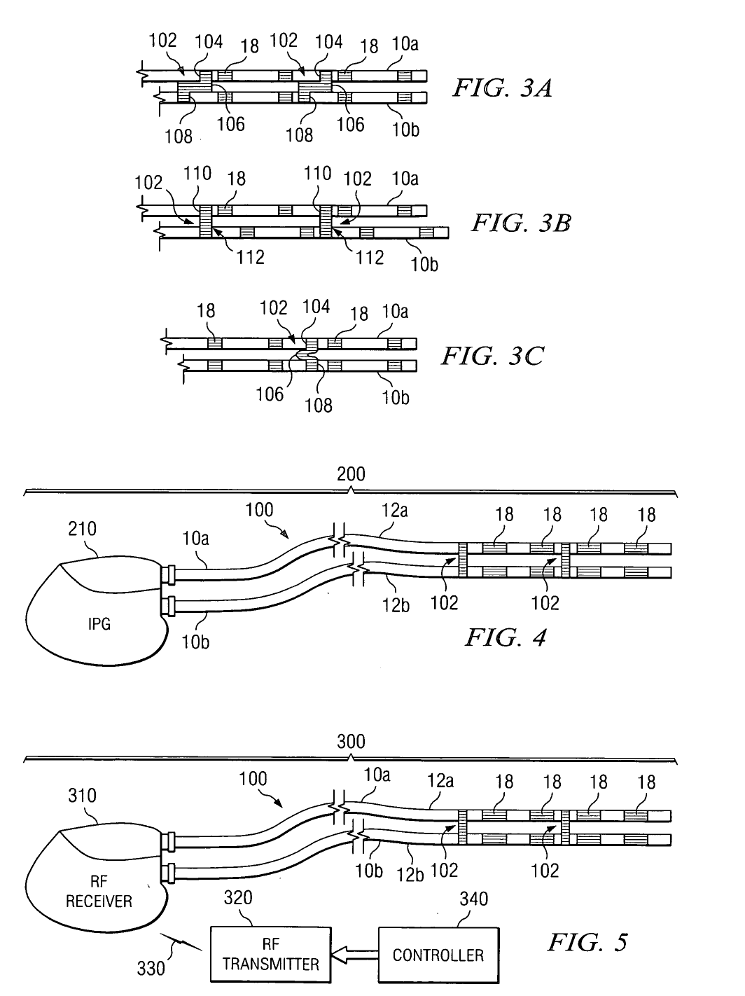

Now referring to FIG. 3A, there is illustrated in detail a portion of the lead 100 of the present invention prior to insertion of the lead 100 into a body (not shown). The leads 10a, 10b are coupled (or attached) relatively close together to allow for insertion of the lead 100 into the body percutaneously via a needle. The connection member 102 connects to each lead 10a, 10b to hold the leads together. Now referring to FIG. 3B, there is illustrated in detail a portion of the lead 100 of the present invention after insertion of the lead 100 into the body (not shown) and deployment. After insertion, the doctor (or other medical professional) deploys the leads into the configuration shown by pulling (or pushing) the two leads apart. The connection member 102 maintains the two leads 10a, 10b at a predetermined distance and/or orientation from one another.

The connection member 102 includes a first portion 104, a second portion 106, and a third portion 108. The first portion 104 is coupled or attached to the second portion 106, and couples or attaches the connection member 102 to the lead 10a. Likewise, the third portion 108 is coupled or attached to the second portion 106, and couples or attaches the connection member 102 to the lead 10b. The portions may be constructed as a unitary structure or one-piece structure, or as separate structures or portions that are coupled or attached together to form the connection member 102. Those skilled in the art will understand that portions 104, 106 , 108 may also be made into one unitary portion, and the dissection into three portions is for illustrative purposes only.

The coupling of portions 104 and 108 may form a hinge, a unitary body, or a different flexible structure such as that shown in FIG. 3C. Those skilled in the art will understand that many different combinations are available that will work as part of the present invention.

FIGS. 3A and 3B illustrate, respectively, one embodiment of the present invention prior to insertion of the leads into a body and after insertion and deployment of the leads. The connection member 102 may be positioned at, or coupled or attached to, different locations along the leads 10a, 10b, such that the electrodes 18 of one lead 10a are positioned as desired relative to the electrodes 18 of the other lead 10b (e.g., in-line or staggered).

Now referring to FIG. 3C, there is illustrated another embodiment of a portion of the lead of the present invention prior to insertion of this lead into a body (a figure illustrating the lead after insertion and deployment is not shown as such illustration would be the same or similar to that shown in FIG. 3B).

In one embodiment, the connection member 102 (or portion thereof) is constructed of resorbable material. Resorbable material is material defined as material that readily dissolves, or is absorbable or resorbable, in a body over a period of time, including such materials identified using the terms absorbable, resorbable or bioabsorbable. Examples of such material include, but are not limited to, catgut suture material(s), polymer materials, polyglycol acid (PGA), polyactic acid (PLA), polydioxanone (PDO), other polymers and polyesters, and any other material that may be known to those skilled in the art exhibiting the desire functioning and characteristics of resorbable material. In one embodiment, the resorbable material has some relative stiffness that functions to separate and sustain a distance between the two leads 10a, 10b.

In another embodiment, at least one portion of the first, second or third portions 104, 106, 108 of the connection member is made of resorbable material. As the leads 10a, 10b are deployed in the body, if one of the portions is absorbable, then when that portion is absorbed, the leads 10a, 10b are separated and are the capable of removal by non-surgical means (the lead is pulled from the body).

Different materials having different resorbable characteristics, such as strength, stiffness, amount of time to resorb, etc., may be used as desired. The specific composition and type of resorbable material utilized will be determined (as necessary or desired) by those skilled in the art, and is likely to be chosen to meet the desired] The present invention lead 100 is shown in FIGS. 2A and 2B with two leads (or lead bodies) 10a, 10b. It will be understood that additional leads (or lead bodies), such as three, four, etc. may be utilized herein consistent with the present invention.

The operation of the present invention will now be described. Prior to insertion, the connection 102 binds or couples the two leads 10a, 10b together (see FIGS. 3A, 3C). The lead 100 is then inserted into a body through an insertion tube or needle at the insertion site. This provides for the two leads 10a, 10b to be inserted at the same time, and with as small a cross-sectional area as practical. Once inserted and placed at the proper location, the leads 10a, 10b are deployed resulting in the fixation (distance and/or orientation, etc.) of one lead 10a relative to the other lead 10b. In one embodiment, the deployment is accomplished by pulling one lead in the proximal direction while the other lead is held in a relatively and substantially fixed position. Alternatively, one of the leads 10a, 10b may be pushed in the distal direction while the other lead is held in relatively fixed position, or one lead may be pulled in one direction (proximal) while the other lead is pulled in the other direction (distal). In other embodiments, the connection member 102 may include resorbable material or structure that provides a spring-type mechanism that deploys the leads after insertion (i.e, when inserted through a tube to the stimulation site, and the tube is removed, the resorbable connection member springs open); a joint to flex open; and/or structure that is forced together during insertion and deploys passively and allows the positioning of the leads relative to each other but limits the distance the leads may spread apart.

The pulling/pushing motion (or disengagement of the connecting member 102) of the leads 10a, 10b, described above, results in the tearing (or breaking) or deployment of a substantial portion of the connection member 102 between the first lead 10a and the second lead 10b. The disengagement or deployment of the connecting member 102 allows the connecting member 102 to maintain the first lead 10a and the second lead 10b in a second substantially fixed location with respect to each other. However, the connection member or mechanism 102 is still attached to the leads 10a, 10b at the connection ends 110, 112. This places the first lead 10a and the second lead 10b in a ladder configuration, and thus maintaining orientation and/or distance between the first lead 10a and the second lead 10b. The connection member 102 is operable to fix a position of the first lead body 10a with respect to a position of the second lead body 10b after the lead 100 is placed within a body. The predetermined distance may be a function of the epidural space and the desired distance between the electrodes 18 of the first lead 10a and the second lead 10b. In another embodiment, the connection member 102 provides a loose fixation point relative to each lead. That is, the positioning of the leads 10a, 10b is adjustable after deployment (allows for some movement) but maintains a maximum distance that the leads can be spaced apart.

Over time, fibrous and/or scar tissue grows and builds up around the leads 10a, 10b. This will tend to maintain the leads' orientation relative to each other. Meanwhile, resorbable material of the connection member 102 dissolves, or is absorbed/resorbed, in the body and the leads 10a, 10b are positioned in the proper location. Since the connection member 102 disappears over time, the leads 10a, 10b can be removed without surgical procedure, such as a lamenectomy. This provides a distinct advantage over other leads (having spaced apart electrodes) inserted (through surgery or otherwise) in the body, as the leads of the present invention may be removed without surgery.

Now referring to FIGS. 4 and 5, there are shown two embodiments of a stimulation system 200, 300 in accordance with the present invention. The stimulation systems generate and apply a stimulus to a tissue or to a certain location of a body. In general terms, the system 200, 300 includes a stimulation or energy source 210, 310 and a lead 100 (with the leads 10a, 10b) for application of the stimulus. The lead 100 shown in FIGS. 4 and 5 is the lead 100 of the present invention.

As shown in FIG. 4, the stimulation system 200 includes the lead 100 that is coupled to the stimulation source 210. In one embodiment, the stimulation source 210 includes an implantable pulse generator (IPG). As is known in the art, an implantable pulse generator (IPG) is capable of being implanted within the body (not shown) that is to receive electrical stimulation from the stimulation source 210. An exemplary IPG may be one manufactured by Advanced Neuromodulation Systems, Inc., such as the Genesis® System, part numbers 3604, 3608, 3609, and 3644.

As shown in FIG. 5, the stimulation system 300 includes the lead 100 that is coupled to the stimulation source 310. The stimulation source 310 includes a wireless receiver (not shown). The stimulation source 310 may also be referred to as a wireless receiver. As is known in the art, the stimulation source 310 comprising a wireless receiver is capable of being implanted within the body (not shown) that is to receive the electrical stimulation from the stimulation source 310. An exemplary stimulation source 310 may be those receivers manufactured by Advanced Neuromodulation Systems, Inc., such as the Renew® System, part numbers 3408 and 3416.

The wireless receiver (not shown) within stimulation source 310 is capable of receiving wireless signals from a wireless transmitter 320. The wireless signals are represented in FIG. 5 by a wireless link symbol 330. The wireless transmitter 320 and a controller 340 are located outside of the body that is to receive electrical stimulation from the stimulation source 310. A user of the stimulation source 310 may use the controller 340 to provide control signals for the operation of the stimulation source 310. The controller 340 provides control signals to the wireless transmitter 320. The wireless transmitter 320 transmits the control signals (and power) to the receiver in the stimulation source 310, and the stimulation source 310 uses the control signals to vary the signal parameters of the electrical signals that are transmitted through lead 100 to the stimulation site. An exemplary wireless transmitter 320 may be those transmitters manufactured by Advanced Neuromodulation Systems, Inc., such as the Renew® System, part numbers 3508 and 3516.

As will be appreciated, the contact electrodes 20 (shown in FIG. 2) are not visible in FIG. 4 (or FIG. 5) because the contact electrodes 20 are situated within a receptacle (not shown) of the stimulation source 210, 310. The contact electrodes 20 are in electrical contact with a generator (not shown) of electrical signals within the stimulation source 210, 310. The stimulation source 210, 310 generates and sends electrical signals via the lead 100 (leads 10a, 10b) to the electrodes 18. Understandably, the electrodes 18 are located at a stimulation site (not shown) within the body that is to receive electrical stimulation from the electrical signals. A stimulation site may be, for example, adjacent to one or more nerves in the central nervous system (e.g., spinal cord). The stimulation source 210, 310 is capable of controlling the electrical signals by varying signal parameters (e.g., intensity, duration, frequency) in response to control signals that are provided to the stimulation source 210, 310.

The stimulation sources 210, 310 are each illustrated with plural connecting means for connecting each of the lead 10a and the lead 10b (of the lead 100) to the stimulation source 210, 310. As will be appreciated, an adaptor (not shown) may be utilized that connects together the proximal ends of the leads 10a, 10b and provides a single connection to the stimulation sources 210, 310, thus allowing the stimulation sources 210, 310 to be configured with a single connector. Alternatively, a lead 100 as illustrated in FIG. 2B may be utilized.

It may be advantageous to set forth definitions of certain words and phrases that may be used within this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and if the term “controller” is utilized herein, it means any device, system or part thereof that controls at least one operation, such a device may be implemented in hardware, firmware or software, or some combination of at least two of the same. It should be noted that the functionality associated with any particular controller may be centralized or distributed, whether locally or remotely.

Although the present invention and its advantages have been described in the foregoing detailed description and illustrated in the accompanying drawings, it will be understood by those skilled in the art that the invention is not limited to the embodiment(s) disclosed but is capable of numerous rearrangements, substitutions and modifications without departing from the spirit and scope of the invention as defined by the appended claims.

1. A lead, comprising:
a first lead body having at least one electrode;
a second lead body having at least one electrode; and
a connection member coupled to the first lead body and the second lead body and operable when the connecting member is in a first state to maintain at least a portion of the first lead body in a first position relative to at least a portion of the second lead body.

2. The lead in accordance with claim 1 wherein at least a portion of the connection member comprises resorbable material.

3. The lead in accordance with claim 1 wherein the first state relates to the lead after insertion into a human body.

4. The lead in accordance with claim 3 wherein at least a portion of the connection member comprises resorbable material.

5. The lead in accordance with claim 1 wherein the connection member is further operable when the connecting member is in a second state to maintain the first lead body in a second position relative to the second lead body.

6. The lead in accordance with claim 5 wherein the second state relates to the lead after insertion into a human body.

7. A lead system, comprising:
a first lead;
a second lead; and
means coupled to the first lead and the second lead for maintaining at least a portion of the first lead in a first position relative to at least a portion of the second lead.

8. The lead system in accordance with claim 7 wherein at least a portion of the means for maintaining comprises resorbable material.

9. The lead system in accordance with claim 8 further comprising:
means for placing at least a portion of the first lead in a second position relative to at least a portion of the second lead.

10. A lead system, comprising:
a first lead;
a second lead; and
a connection member, comprising,
a first portion attached to the first lead,
a second portion attached to the second lead and coupled to the first portion, and
and wherein at least one of the first portion and the second portion comprises resorbable material.

11. The lead system in accordance with claim 10 wherein the first portion and the second portion are coupled using a third portion.

12. The lead system in accordance with claim 11 wherein the third portion comprises resorbable material.

13. The lead system in accordance with claim 10 wherein the connection member orients the first lead with respect to the second lead.

14. The lead system in accordance with claim 10 wherein the connection member is operable to maintain a predetermined maximum distance between the first lead and the second lead prior to when the at least one of the first portion and the third portion comprising resorbable material resorbs in a body.

15. The lead system in accordance with claim 10 wherein the third portion comprises resorbable material.

16. The lead system in accordance with claim 10 wherein the connection member couples the first lead to the second lead in a first fixed relation prior to insertion of the lead into a body and in a second fixed relation after insertion of the lead into the body.

17. A lead system, comprising:
a first lead;
a second lead; and
a connection member, comprising,
a first portion attached to the first lead,
a second portion attached to the second lead, and
a third portion coupled to the first portion and the second portion, and
and wherein at least one of the first portion, the second portion and the third portion comprises resorbable material.

18. A lead system, comprising:
a first lead body, comprising,
a proximal end and a distal end,
at least one contact electrode positioned proximate the proximal end,
at least one electrode positioned proximate the distal end, and
at least one conductor extending through the lead body and electrically connecting the contact electrode and the electrode;
a second lead body, comprising,
a proximal end and a distal end,
at least one contact electrode positioned proximate the proximal end,
at least one electrode positioned proximate the distal end, and
at least one conductor extending through the lead body and electrically connecting the contact electrode and the electrode; and
a connection member, comprising,
a first portion attached to the distal end of the first lead body,
a second portion attached to the distal end of the second lead body, and
a third portion coupled to the first portion and the second portion,
and wherein at least one of the first portion, the second portion, and the third portion comprises resorbable material.

19. The lead in accordance with claim 18 wherein the member is operable to maintain the first lead body and the second lead body in a substantially fixed position with respect to each other.

20. A method of inserting and positioning a medical lead within a body, comprising:
inserting a distal end of a lead into a human body, the lead comprising,
a first lead body,
a second lead body, and
a connection member;
after inserting the lead into the human body, disengaging the connection member to allow the connection member to maintain the first lead body and the second lead body in a first position with respect to each other.

21. The method in accordance with claim 20 wherein at least a portion of the connection member comprises resorbable material, and after a period of time, the portion of the connection member comprising resorbable material resorbs into the human body.

22. A method of manufacturing a lead, comprising:
providing a first lead body having a distal end;
providing a second lead body having a distal end;
coupling the distal end of the first lead body to the distal end of the second lead body with a connection member, at least a portion of the connection member comprising resorbable material.

23. The method in accordance with claim 22 wherein the connection member comprises a first portion, a second portion, and a third portion, the first portion coupled o the second portion and the second portion coupled to the third portion, and further comprising:
coupling the first portion to the distal end of the first lead body; and
coupling the third portion of the distal end of the second lead body.

24. A system for stimulating a portion of a body, the system comprising:
a source for generating a stimulus; and
an implantable lead for receiving the stimulus from the source, the implantable lead comprising,
a first lead;
a second lead; and
a connection member, comprising,
a first portion attached to the first lead,
a second portion attached to the second lead, and
a third portion coupled to the first portion and the second portion,
and wherein at least one of the first portion, the second portion, and the third portion comprises resorbable material.

25. The system in accordance with claim 24 wherein the position of the first lead is substantially fixed with respect to the position of the second lead after the lead is inserted within a body.

26. The system in accordance with claim 24 wherein the source comprises a wireless receiver.

27. The system in accordance with claim 24 wherein the source comprises an implantable pulse generator.

28. The system in accordance with claim 24 further comprising a controller operable for communicating with the source and controlling the source.
